(19) United States
(12) Patent Application Publication
(10) Pub. No.: US 2010/0227367 A1
(43) Pub. Date: Sep. 9, 2010

(54) Process of Producing a Fermentation Product

(75) Inventors: Eder Manzini Bordin, Curitiba (BR); Viviane Pereira De Souza, Curitiba-Parana (BR); Fabiane Bueno Ormerod, Curitiba-PR (BR); Adauto De Alameida, JR., Curitiba-Parana (BR)

Correspondence Address: NOVOZYMES NORTH AMERICA, INC., 500 FIFTH AVENUE, SUITE 1600, NEW YORK, NY, 10110, US

(73) Assignees: NOVOZYMES A/S, Bagsvaerd (DK); NOVOZYMES NORTH AMERICA, INC., Franklinton, NC (US)

(21) Appl. No.: 12/671,633

(22) PCT Filed: Oct. 10, 2008
(86) PCT No.: PCT/US08/79490

§ 371 Date: Mar. 16, 2010

Related U.S. Application Data
(60) Provisional application No. 60/979,471, filed on Oct. 12, 2007.

(51) Int. Cl. C12P 7/06

(52) U.S. Cl. 435/161

(57) ABSTRACT

The invention relates to a process for producing a fermentation product from molasses wherein molasses is i) treated with a combination of alpha-amylase and glucoamylase and ii) fermented using one or more fermenting organisms at a cell count in the range from 10⁷-10¹⁰ cells/mL fermentation medium.

### FIELD OF THE INVENTION

The present invention relates to processes for producing a fermentation product, such as ethanol, from molasses.

### BACKGROUND OF THE INVENTION

Large scale commercial production of fuel ethanol from molasses is known in the art. Molasses is a by-product of sugar cane or sugar beet refining. Molasses is a dark-brown sweet syrup containing about 50% sucrose. When juices extracted from sugar cane or sugar beet is evaporated the removal of water facilitates the separation of sugar in crystalline form. When this process of sugar crystallization has reached its limit, and the sugar crystals are removed, the remaining dark brown thick syrup is known as molasses.

WO 96/13600 discloses a method to produce fermentable mono-saccharides from un-fermentable saccharides, present in liquefied and/or saccharified starch, beet molasses and sugar cane molasses, in order to improve the raw material utilization in fermentation processes such as fermentative production of ethanol.

U.S. Pat. No. 4,769,324 is directed to the production of ethanol by fermentation of molasses in the presence of yeast which is capable of growing and producing amylase in a molasses-containing medium.

BR-PI-990252-8-A discloses a process of producing ethanol wherein fermenting yeast is deflocculated by enzymatic action of protease or enzymes such as glucanases, cellulases, chitinases, xylanases, and acid or alkaline laminarinases.

There is a need for further improvement of fermentation product, such as ethanol, manufacturing processes.

### SUMMARY OF THE INVENTION

The invention relates to processes for producing fermentation products from molasses using a fermenting organism, wherein molasses is
i) treated with a combination of alpha-amylase and glucoamylase, and
ii) fermented using one or more fermenting organisms at a cell count in the range from 10⁷-10¹¹ cells/mL fermentation medium.

According to the invention the feedstock is molasses which is a by-product of, e.g., sugar cane or sugar beet refining.

### BRIEF DESCRIPTION OF THE DRAWINGS

FIG. 1 shows the °Bx development during fermentation during molasses fermentation.

FIG. 2 shows the pH development during molasses fermentation for two enzyme blends containing alpha-amylase, glucoamylase and protease added during simultaneous saccharification and fermentation.

FIG. 3 shows the °Bx linear trend line for an enzyme blend containing alpha-amylase, glucoamylase and protease added during simultaneous saccharification and fermentation.

FIG. 4 shows the pH development during molasses fermentation for two enzyme blends containing alpha-amylase and glucoamylase added during simultaneous saccharification and fermentation.

FIG. 5 shows the °Bx development during molasses fermentation for two enzyme blends containing alpha-amylase and glucoamylase added during simultaneous saccharification and fermentation.

FIG. 6 shows the °Bx linear trend lines for two enzyme blends containing alpha-amylase and glucoamylase added during simultaneous saccharification and fermentation.

FIG. 7 shows the ethanol yield after 30 hours enzymatic pre-treatment of molasses followed by 6 hours fermentation.

FIG. 8 shows the ethanol yield after 30 hours enzymatic pre-treatment of molasses followed by 10 hours fermentation.

FIG. 9 shows the productivity gain as total reducing sugar (TRS) decay after enzymatic pre-treatment followed by 6 hours fermentation.

FIG. 10 shows the productivity gain as total reducing sugar (TRS) decay after enzymatic pre-treatment followed by 10 hours fermentation.

FIG. 11 shows the viscosity during simultaneous saccharification and fermentation with enzymes blends.

### DETAILED DESCRIPTION OF THE INVENTION

The present invention provides processes for producing a fermentation product, especially ethanol, from molasses using a fermenting organism.

The inventors have found that when subjecting molasses to a combination of alpha-amylase and glucoamylase the productivity is increased. This is advantageous as the fermentation time can be shortened. Without being limited by any theory it is believed that treatment with alpha-amylase and glucoamylase results in a viscosity and/or density reduction in the fermentation medium. This way the influx of fermentable sugars in the fermentation medium over the fermenting organism's cell membrane is facilitated. This may result in an increase in the sugars-to-fermentation product conversion rate leading to shortened fermentation time and thus higher productivity. An alternative or additional theory is that the cell concentration and/or cell viability is increased. The inventors also found that when pre-treating molasses before carrying out fermentation a yield improvement may be obtained.

The invention relates to processes for producing fermentation products from molasses using a fermenting organism, wherein molasses is
i) treated with a combination of alpha-amylase and glucoamylase, and
ii) fermented using one or more fermenting organisms at a cell count in the range from 10⁷-10¹¹ cells/mL fermentation medium.

In a preferred embodiment the cell count is in the range 10⁸-10¹⁰ cells/mL fermentation medium, especially around 10⁹ cells/mL fermentation medium.

Concentrated molasses has a °Bx around 80%. In the fermentation medium the molasses is diluted in water so that the molasses during a process of the invention has a °Bx in the range from around 1-35%, preferably 16-25%, preferably around 18-22%. In high gravity processes the °Bx is in the range from in the range from 25-35, preferably 27-32°Br

Brix (°Bx) is a measurement of the mass ratio of dissolved solids (e.g., sucrose) to water in a liquid (e.g., water). It may be measured with equipment (e.g., saccharimeter) that measures specific gravity of a liquid. For instance, a 25°Bx solution is 25% (w/w), with 25 grams of sucrose sugar per 100 grams of liquid, i.e., there are 25 grams of sucrose sugar and 75 grams of water in the 100 grams of solution.

The enzyme treatment in step i) and fermentation in step ii) may be carried out either sequentially or simultaneously. In a preferred embodiment, where the steps are carried out sequentially, the enzyme treatment step i) is carried out as a pre-treatment step, preferably at conditions suitable for the enzymes. In an embodiment step i) is carried out at a temperature in the range from 20-70° C., preferably 40-60° C., preferably 45-55° C. The pH during treatment is preferably in the range from 4-S. The pre-treatment in step i) may be carried out for between 1-10 days, followed by fermentation for 1-80 hours, preferably 1-70 hours or 1-15 hours, such as 1-10 hours.

In an embodiment molasses (°Br around 80%) is pre-treated in a surge tank at 40-60° C. for 1-10 days at a pH in the range from 4-6. The pre-treated molasses is thereafter fermented at a °Br in the range 16-24%, pH 3-6 at a temperature between 30-36° C. for 1-18 hours or 1-15 hours.

When the process of the invention is carried out as a simultaneous step i) and step ii) process the temperature range used is suitable, preferably optimal, for the fermenting organism(s). The temperature depends on the fermenting organisms in question. In a preferred embodiment the temperature lies in the range from 25-60° C. One skilled in the art can easily determine the suitable or optimal temperature. The process time is in one embodiment in the range from about 1 to 96 hours, preferably between 5 and 72 hours.

In an embodiment molasses (°Br 16-24%) is fermented at a temperature in the range 30-36° C., pH 3-6, for 6-96 hours.

If the process of the invention is an ethanol production process using yeast, such as a strain of Saccharomyces, preferably a strain of Saccharomyces cerevisiae, as the fermenting organism the process may preferably be carried out at a temperature from 25-40° C., preferably from 28-36° C., especially in the range from 30-34° C., such as around 32° C.

In a further embodiment a protease is also present during the process of the invention. In an embodiment the protease is added during enzyme treatment in step i) or during simultaneous enzyme treatment and fermentation. The protease may be added to in order to deflocculate the fermenting organism, especially yeast, during fermentation.

### Fermentation

The term “fermenting organism” refers to any organism suitable for use in a desired fermentation process. Suitable fermenting organisms are according to the invention capable of fermenting, i.e., converting, preferably DP₁₋₃ sugars, such as especially glucose, fructose and maltose, directly or indirectly into the desired fermentation product, such as ethanol. The fermenting organism is typically added to the mash.

Examples of fermenting organisms include fungal organisms, such as yeast or filamentous fungi. Preferred yeast includes strains of the Saccharomyces spp., and in particular Saccharomyces cerevisiae. Commercially available yeast includes, e.g., RED STAR®/Lesaffre Ethanol Red (available from Red Star/Lesaffre, USA), FALI (available from Fleischmann's Yeast, USA) SUPERSTART (available from Alltech), GERT STRAND (available from Gert Strand AB, Sweden) and FERMIOL (available from DSM Specialties).

Preferred yeast for ethanol production includes, e.g., Pichia and Saccharomyces. Preferred yeast according to the invention is Saccharomyces species, in particular Saccharomyces cerevisiae or bakers yeast.

### Recovery

The process of the invention may optionally comprise recovering the fermentation product, such as ethanol; hence the fermentation product, e.g., ethanol, may be separated from the fermented material and purified. Following fermentation, the mash may be distilled to extract, e.g., ethanol. Ethanol with a purity of up to, e.g., about 96 vol. % ethanol can be obtained by the process of the invention.

Thus, in one embodiment, the fermentation in step ii) and a distillation step may be carried out simultaneously and/or separately/sequentially; optionally followed by one or more process steps for further refinement of the fermentation product, e.g., ethanol.

### Enzymes

### Alpha-Amylase

According to the invention any alpha-amylase may be used in a process of the invention. Preferred alpha-amylases are of microbial, such as bacterial or fungal origin. In one embodiment the preferred alpha-amylase is an acid alpha-amylase, e.g., fungal acid alpha-amylase or bacterial acid alpha-amylase. The term “add alpha-amylase” means an alpha-amylase (E.C. 3.2.1.1) which when used in a process of the invention has an activity optimum at a pH in the range from 3 to 7, preferably from 3.5 to 6, or more preferably from 4-5.

### Bacterial Alpha-Amylase

In an embodiment the alpha-amylase is of Bacillus origin. A Bacillus alpha-amylase may preferably be derived from a strain of B. licheniformis, B. amyloliquefaciens, B. subtilis or B. stearothermophilus, but may also be derived from other Bacillus sp. strains. Specific examples of contemplated alpha-amylases include the Bacillus licheniformis alpha-amylase shown in SEQ ID NO: 4 in WO 99/19467, the Bacillus amyloliquefaciens alpha-amylase SEQ ID NO: 5 in WO 99/19467 and the Bacillus stearothermophilus alpha-amylase shown in SEQ ID NO: 3 in WO 99/19467 (all sequences hereby incorporated by reference). In an embodiment of the invention the alpha-amylase may be an enzyme having a degree of identity of at least 60%, preferably at least 70%, more preferred at least 80%, even more preferred at least 90%, such as at least 95%, at least 96%, at least 97%, at least 98% or at least 99% to any of the sequences shown in SEQ ID NOS: 1, 2 or 3, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 or U.S. Pat. No. 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta (181-182) compared to the wild-type BSG alpha-amylase amino acid sequence set forth in SEQ ID NO:3 disclosed in WO 99/19467 or deletion of amino acids R179 and G180 using SEQ ID NO:3 in WO 99/19467 for numbering (which reference is hereby incorporated by reference). Even more preferred are Bacillus alpha-amylases, especially Bacillus stearothermophilus alpha-amylase, which have a double deletion corresponding to delta (181-182) and further comprise a N193F substitution (also denoted I181*+G182*+N193F) compared to the wild-type BSG alpha-amylase amino acid sequence set forth in SEQ ID NO:3 disclosed in WO 99/19467.

### Bacterial Hybrid Alpha-Amylase

Hybrid alpha-amylases specifically contemplated comprise 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO; 5 of WO 99/19467), with one or more, especially all, of the following substitution:

G48A+T49I+G107A+H 156Y+A 181T+N190F+1201F+A209V+Q264S (using the Bacillus licheniformis numbering in SEQ ID NO: 4 of WO 99/19467). Also preferred are variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using the SEQ ID NO: 5 numbering of WO 99/19467).

Bacterial alpha-amylase may be added in concentrations well-known in the art. When measured in KNU units (described below in the Materials & Methods”-section) the alpha-amylase activity is preferably present in the range from 0.5-50 KNU/L fermentation medium, such as 1-25 KNU/L fermentation medium, or more preferably in an amount of 2-10 KNU/L fermentation medium.

### Fungal Alpha-Amylase

Fungal alpha-amylases include alpha-amylases derived from a strain of the genus Aspergillus, such as, from a strain of Aspergillus oryzae, Aspergillus niger and Aspergillis kawachii.

Preferred acid fungal alpha-amylases include Fungamyl-like alpha-amylases which are derived from a strain of Aspergillus, preferably Aspergillus oryzae. According to the present invention, the term “Fungamyl-like alpha-amylase” indicates an alpha-amylase which exhibits a high identity, i.e., at least 70%, at least 75%, at least 80%, at least 85%, at least 90%, at least 95%, at least 96%, at least 97%, at least 98%, even at least 99% or even 100% identity to the mature part of the amino acid sequence shown in SEQ ID NO: 10 in WO 96/23874.

Other preferred acid alpha-amylases are derived from a strain of Aspergillus niger. In a preferred embodiment the acid fungal alpha-amylase is the one from Aspergillus niger disclosed as “AMYA_ASPNG” in the Swiss-prot/TeEMBL database under the primary accession no. P56271 and described in WO 89/01969 (Example 3). A commercially available acid fungal alpha-amylase derived from Aspergillus niger is SP288 (available from Novozymes A/S, Denmark). Other contemplated wild-type alpha-amylases include those derived from strains of the genera Rhizomucor and Meripilus, preferably a strain of Rhizomucor pusillus (WO 2004/055178 incorporated by reference) or Meripilus giganteus.

In a preferred embodiment the alpha-amylase is derived from Aspergillus kawachii and disclosed by Kaneko et al. J. Ferment. Bioeng. 81:292-298 (1996) “Molecular-cloning and determination of the nucleotide-sequence of a gene encoding an acid-stable alpha-amylase from Aspergillus kawachii”; and further as EMBL:#AB008370.

The fungal alpha-amylase may also be a wild-type enzyme comprising a starch-binding domain (SBD) and an alpha-amylase catalytic domain (i.e., none-hybrid), or a variant thereof. In an embodiment the wild-type alpha-amylase is derived from a strain of Aspergillus kawachii.

### Fungal Hybrid Alpha-Amylase

In a preferred embodiment the fungal acid alpha-amylase is a hybrid alpha-amylase. Preferred examples of fungal hybrid alpha-amylases include the ones disclosed in WO 2005/003311 or U.S. Patent Publication no. 2005/0054071 (Novozymes) or US patent application No. 60/638,614 (Novozymes) which is hereby incorporated by reference. A hybrid alpha-amylase may comprise an alpha-amylase catalytic domain (CD) and a carbohydrate-binding domain/module (CBM), such as a starch binding domain, and optional a linker.

Specific examples of contemplated hybrid alpha-amylases include those disclosed in Table 1 to 5 of the examples in U.S. patent application No. 60/638,614, including Fungamyl variant with catalytic domain JA118 and Athelia rolfsii SBD (SEC) ID NO:100 in U.S. 60/638,614), Rhizomucor pusillus alpha-amylase with Athelia rolfsii AMG linker and SBD (SEQ ID NO:101 in U.S. 60/638,614), Rhizomucor pusillus alpha-amylase with Aspergillus niger glucoamylase linker and SBD (which is disclosed in Table 5 as a combination of amino acid sequences SEQ ID NO:20 SEQ ID NO:72 and SEQ ID NO:96 in U.S. application Ser. No. 11/316,535 and further as SEQ ID NO: 13 herein) or as V039 in Table 5 in WO 2006/069290, and Meripilus giganteus alpha-amylase with Athelia rolfsii glucoamylase linker and SBD (SEQ ID NO:102 in U.S. 60/638,614). Other specifically contemplated hybrid alpha-amylases are any of the ones listed in Tables 3, 4, 5, and 6 in Example 4 in U.S. application Ser. No. 11/316,535 and WO 2006/069290 (hereby incorporated by reference).

Other specific examples of contemplated hybrid alpha-amylases include those disclosed in U.S. Patent Publication no. 2005/0054071, including those disclosed in Table 3 on page 15, such as Aspergillus niger alpha-amylase with Aspergillus kawachii linker and starch binding domain.

Contemplated are also alpha-amylases which exhibit a high identity to any of above mention alpha-amylases, i.e., at least 70%, at least 75%, at least 80%, at least 85%, at least 90%, at least 95%, at least 96%, at least 97%, at least 98%, at least 99% or even 100% identity to the mature enzyme sequences.

An acid alpha-amylases may be added in an amount of 0.1 to 250 FAU(F)/L fermentation medium, preferably 1 to 100 FAU(F)/L fermentation medium.

### Commercial Alpha-Amylase Products

Preferred commercial compositions comprising alpha-amylase include MYCOLASE™ from DSM, BAN™, TERMAMYL™ SC, FUNGAMYL™, LIQUOZYME™ X and SAN™ SUPER, SAN™, EXTRA L (Novozymes A/S) and CLARASE™ L-40,000, DEX-LO™, SPEZYME™ FRED, SPEZYME™ AA, and SPEZYME™ DELTA AA (Genencor Int.), and the acid fungal alpha-amylase sold under the trade name SP288 (available from Novozymes A/S, Denmark).

### Glucoamylase

A glucoamylase used according to the process of the invention may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, A. oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen at al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka, Y. et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215) or Trametes cingulata disclosed in WO 2006/069289 (which is hereby incorporated by reference).

Bacterial glucoamylases contemplated include glucoamylases from the genus Clostridium, in particular C. thermoamylolyticum (EP 135,138), and C. thermohydrosulfuricum (WO 86/01831) (which is hereby incorporated by reference).

Also hybrid glucoamylase are contemplated according to the invention. Examples the hybrid glucoamylases disclosed in WO 2005/045018. Specific examples include the hybrid glucoamylase disclosed in Table 1 and 4 of Example 1 (which hybrids are hereby incorporated by reference.).

Contemplated are also glucoamylases which exhibit a high identity to any of above mention glucoamylases, i.e., at least 70%, at least 75%, at least 80%, at least 85%, at least 90%, at least 95%, at least 96%, at least 97%, at least 98%, at least 99% or even 100% identity to the mature enzymes sequences.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int).

Glucoamylases may in an embodiment be added in an amount of 1-5,000 AGU/L fermentation medium, preferably 10-1,000 AGU/L fermentation medium.

### Proteases

The protease may be any protease. In a preferred embodiment the protease is an acid protease of microbial origin, preferably of fungal or bacterial origin. An acid fungal protease is preferred, but also other proteases can be used.

Using protease in a process of the invention generally reduces flocculation of fermenting organism cells, especially yeast cells, and also results in an increase in the FAN (Free Amino Nitrogen) level which leads to an increase in fermenting organism's metabolism.

Suitable proteases include microbial proteases, such as fungal and bacterial proteases. Preferred proteases are acidic proteases, i.e., proteases characterized by the ability to hydrolyze proteins under acidic conditions below pH 7.

Contemplated acid fungal proteases include fungal proteases derived from Aspergillus, Mucor, Rhizopus, Candida, Coriolus, Endothia, Enthomophtra, Irpex, Penicillium, Scierotiumand Torulopsis. Especially contemplated are proteases derived from Aspergillus niger (see, e.g., Koaze et al., (1964), Agr. Biol. Chem. Japan, 28, 216), Aspergillus saitoi (see, e.g., Yoshida, (1954) J. Agr. Chem. Soc. Japan, 28, 66), Aspergillus awamori (Hayashida et al., (1977) Agric. Biol, Chem., 42(5), 927-933, Aspergillus aculeatus (WO 95/02044), or Aspergillus oryzae, such as the pepA protease; and acidic proteases from Mucor pusillus or Mucor miehei.

Contemplated are also neutral or alkaline proteases, such as a protease derived from a strain of Bacillus. A particular protease contemplated for the invention is derived from Bacillus amyloliquefaciens and has the sequence obtainable at Swissprot as Accession No. P06832. Also contemplated are the proteases having at least 90% identity to amino acid sequence obtainable at Swissprot as Accession No. P06832 such as at least 92%, at least 95%, at least 96%, at least 97%, at least 98%, or particularly at least 99% identity.

Further contemplated are the proteases having at least 90% identity to amino acid sequence disclosed as SEQ. ID. NO: 1 in the WO 2003/048353 such as at 92%, at least 95%, at least 96%, at least 97%, at least 98%, or particularly at least 99% identity.

Also contemplated are papain-like proteases such as proteases within E.C. 3.4.22.* (cysteine protease), such as EC 3.4.22.2 (papain), EC 3.4.22.6 (chymopapain), EC 3.4.22.7 (asclepain), EC 3.4.22.14 (actinidain), EC 3.4.22.15 (cathepsin L), EC 3.4.22.25 (glycyl endopeptidase) and EC 3.4.22.30 (caricain).

In an embodiment the protease is a protease preparation derived from a strain of Aspergillus, such as Aspergillus oryzae. In another embodiment the protease is derived from a strain of Rhizomucor, preferably Rhizomucor mehei. In another contemplated embodiment the protease is a protease preparation, preferably a mixture of a proteolytic preparation derived from a strain of Aspergillus, such as Aspergillus oryzae, and a protease derived from a strain of Rhizomucor, preferably Rhizomucor mehei.

Aspartic acid proteases are described in, for example, Hand-book of Proteolytic Enzymes, Edited by A. J. Barrett, N. D. Rawlings and J. F. Woessner, Aca-demic Press, San Diego, 1998, Chapter 270). Suitable examples of aspartic acid protease include, e.g., those disclosed in R. M. Berka at al. Gene, 96, 313 (1990)); (R. M. Berke et al. Gene, 125, 195-198 (1993)); and Gomi at al. Biosci. Biotech, Biochem. 57, 1095-1100 (1993), which are hereby incorporated by reference.

Commercially available products include ALCALASE™, ESPERASE™, FLAVOURZYME™, PROMIX™, NEUTRASE®, RENNILASE®, NOVOZYM™ FM 2.0 L, and NOVOZYM™ 50006 (available from Novozymes A/S, Denmark) and GC106™ and SPEZYME™ FAN from Genencor Int., Inc., USA.

The protease may be present in an amount of 0.001-1 AU/L fermentation medium, preferably 0.005 to 0.5 AU/L fermentation medium, especially 0.05-0.1 AU/L fermentation medium.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control. Various references are cited herein, the disclosures of which are incorporated by reference in their entireties. The present invention is further described by the following examples which should not be construed as limiting the scope of the invention.

### Material & Methods

### Enzymes:

Protease ALC: Wild-type alkaline protease derived from Bacillus licheniformis available from Novozymes A/S, Denmark.
Glucoamylase SF: Glucoamylase derived from Talaromyces emersonii and disclosed as SEQ ID NO: 7 in WO 99/28448.
Glucoamylase TC: Glucoamylase derived from Trametes cingulate disclosed in SEQ ID NO: 2 in WO 2006/069289 and available from Novozymes A/S, Denmark.
Alpha-amylase SC: Bacillus stearothermophilus alpha-amylase variant with the mutations: I181*+G182*+N193F disclosed in U.S. Pat. No. 6,187,576 and available on request from Novozymes A/S, Denmark.
Alpha-Amylase JA: Hybrid alpha-amylase consisting of Rhizomucor pusillus alpha-amylase with Aspergillus niger glucoamylase linker and SBD disclosed as V039 in Table 5 in WO 2006/069290 (Novozymes A/S).

Stored Molasses: Sugar Case molasses stored since 2006 obtained from City of Aracatuba, San Paolo State, Brazil.

Fresh Molasses: Sugar Cane molasses produced in 2007 obtained from City of Lencoes Paulista, Sao Paolo State, Brazil.

### Determination of Identity

The term “identity” means the degree of identity between two amino acid sequences. The homology may suitably be determined by computer programs known in the art, such as, GAP provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-453. The following settings for polypeptide sequence comparison are used: GAP creation penalty of 3.0 and GAP extension penalty of 0.1.

### Alpha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha-amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum soluble.

A folder EB-SM-0009.02/01 describing this analytical method in more detail is available upon request to Novozymes A/S, Denmark, which folder is hereby included by reference.

### Determination of FAU(F)

FAU(F) Fungal Alpha-Amylase Units (Fungamyl) is measured relative to an enzyme standard of a declared strength.

Reaction conditions

| | |
|---|---|
| Temperature | 37° C. |
| pH | 7.15 |
| Wavelength | 405 nm |
| Reaction time | 5 min |
| Measuring time | 2 min |

A folder (EB-SM-0216.02) describing this standard method in more detail is available on request from Novozymes A/S, Denmark, which folder is hereby included by reference.

### Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| | |
|---|---|
| AMG incubation: | |
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |
| Color reaction: | |
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this: analytical method in more detail is available on request from Novozymes A/S, Denmark, which folder is hereby included by reference.

### Protease Assay Method—AU(RH)

The proteolytic activity may be determined with denatured hemoglobin as substrate. In the Anson-Hemoglobin method for the determination of proteolytic activity denatured hemoglobin is digested, and the undigested hemoglobin is precipitated with trichloroacetic acid (TCA). The amount of TCA soluble product is determined with phenol reagent, which gives a blue color with tyrosine and tryptophan.

One Anson Unit (AU-RH) is defined as the amount of enzyme which under standard conditions (i.e. 25° C., pH 5.5 and 10 min. reaction time) digests hemoglobin at an initial rate such that there is liberated per minute an amount of TCA soluble product which gives the same color with phenol reagent as one milliequivalent of tyrosine.

The AU(RH) method is described in EAL-SM-0350 and is available from Novozymes A/S Denmark on request.

### EXAMPLES

### Example 1

### Simultaneous Saccharification and Fermentation of Sugar Cane Molasses

This example investigates the effect of alpha-amylase, glucoamylase and protease in an ethanol fermentation process using sugar-cane molasses as feedstock.

Stored sugar-cane molasses was diluted in tap water to a °Bx of 18-20%. The pH was adjusted to 4.7-4.9 with sulfuric acid. The diluted molasses was filled into approximately 25 mL tubes with caps. The fermentation medium was not supplemented with nitrogen, phosphate, vitamin or antibiotic.

Yeast inoculum was prepared in a °Bx 5-7% molasses solution. The Saccharomyces cerevisiae yeast (RED STAR™) inoculum was added to the fermentation medium until the suspension contained about 40-50% solids (corresponding to between 10⁸-10⁹ cells/mL fermentation medium) measured using a centrifuge (2500 rpm, 20° C. for 10 minutes). The yeast suspension was incubated at room temperature (18-25° C.) for around 12 hours.

Enzymes were diluted in tap water and pipetted into the tubes and homogenized.

Dosage and Enzymes Used:

Alpha-amylase SC: 9.6 KNU/L fermentation medium
Glucoamylase SF: 60 AGU/L fermentation medium
Protease ALC: 0.048 AU/L fermentation medium

Fermentation was initiated by adding 2 mL of yeast suspension into the tubes. All tubes were incubated in a water bath at 32±0.5° C. for 24 hours. The experiment was set up with 5 tubes for each treatment (duplicate).

The following analyses were carried out: pH (potentiometer), % Brix (refractometer), viscosity (viscometer. ANTO PAAR, DMA 5000 and micro-viscometer AMVn) and HPLC (AMEX HPX-87H, 0.005M Sulfuric acid, 65° C. temp, 10 microL injection volume and 30 min of run time).

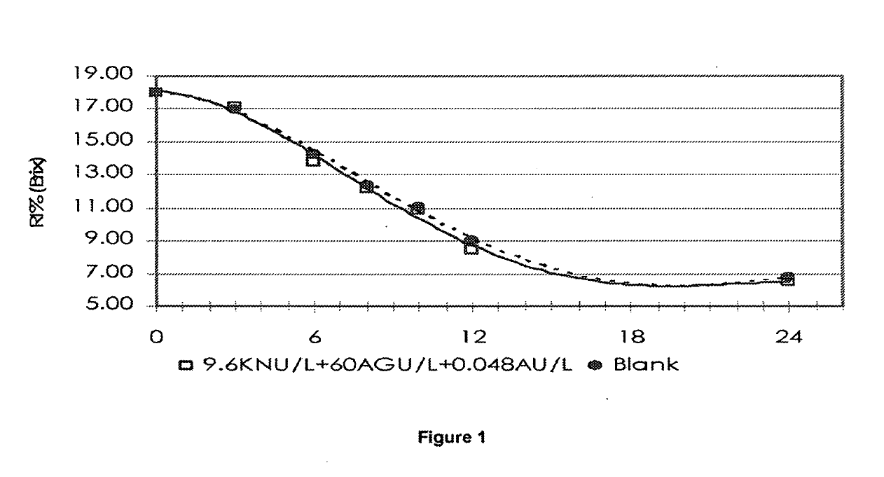

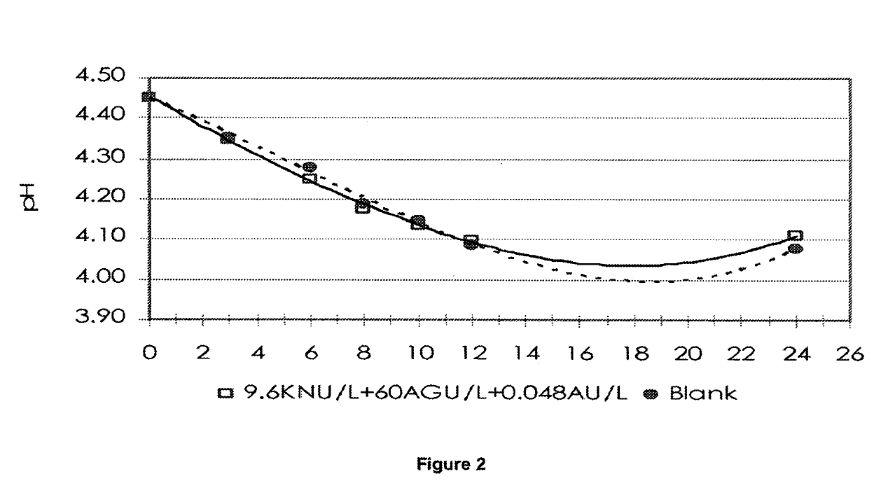

The blank (no enzymes added) was compared with enzymatic treatment with 9.6 KNU/L+60 AGU/L+0.048 AU/L fermentation medium. The results are shown in FIGS. 1 and 2.

In general, °Bx decay measures the consumption of fermentable sugar by the yeast.

pH gives an indication of the contamination. Normally acids are produced by contaminants which reduce the pH. pH increase could mean starvation of the yeast as a consequence of lack of nutrients.

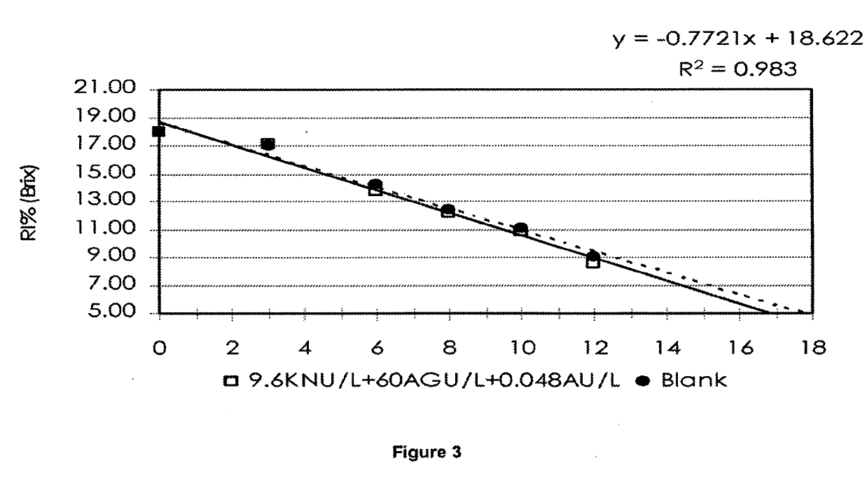

When °Bx is steady for at least 1 hour the fermentation is considered finalized. The trail showed that fermentation of enzymatically treated molasses was finalized before the blank (control). The productivity gain is estimated to be around 6% as demonstrated by °Bx linear trend line shown in FIG. 3.

### Example 2

### Simultaneous Saccharification and Fermentation of Solar Cane Molasses

This example was carried out at the same experimental condition as in Example 1. Below dosages and enzyme blends were used.
Alpha-amylase SC (19 KNU/L)+Glucoamylase SF (120 AGU/L);
Alpha-Amylase JA (26 FAU(F)/L)+Glucoamylase TC (160 AGU/L);

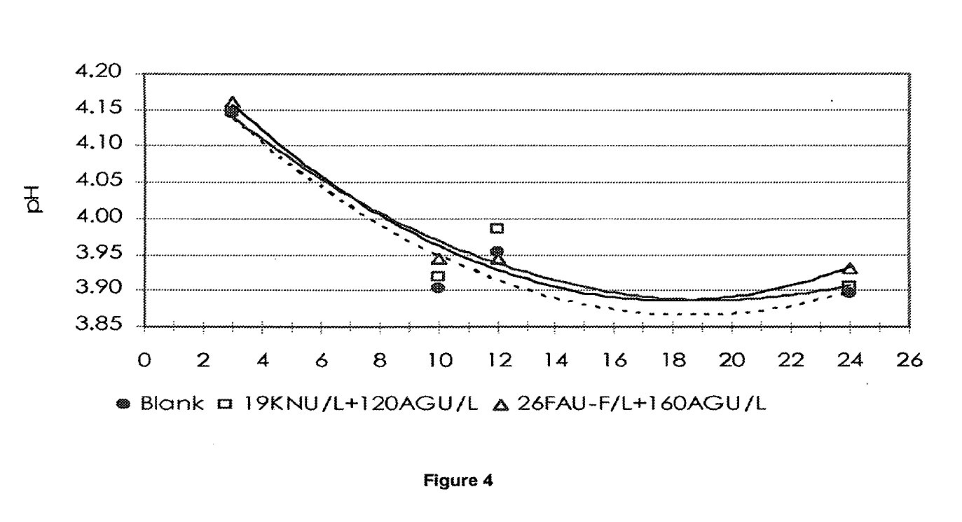

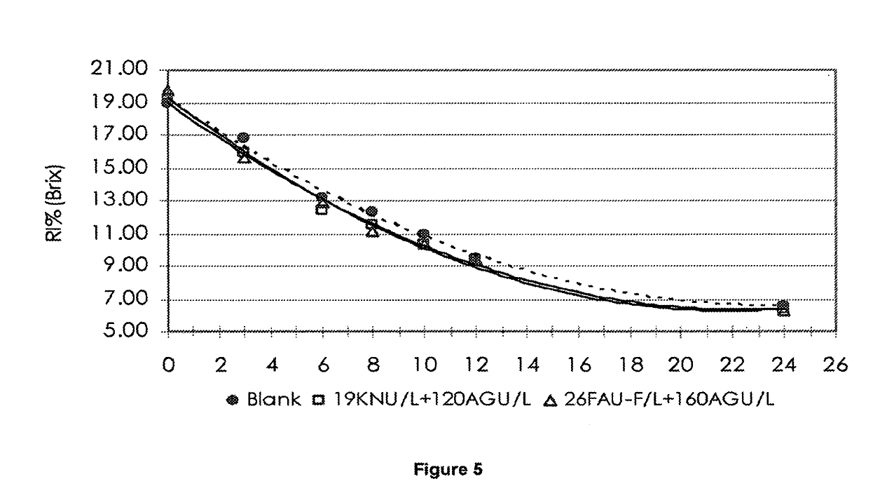

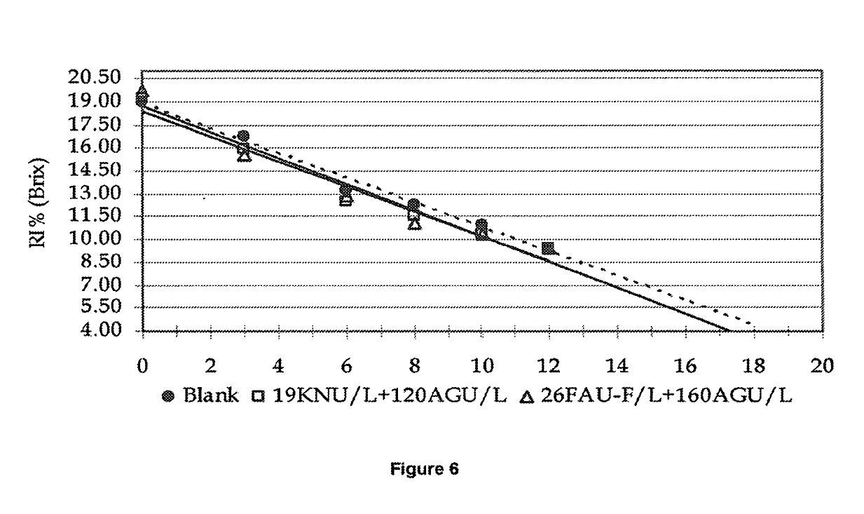

FIGS. 4 and 5, respectively, display the pH and °Bx decay curves for above blends. The productivity gain is estimated to be around 6% as demonstrated by the °Bx linear trend line shown in FIG. 6.

### Example 3

### Enzymatic Pre-Treatment of Sugar Cane Molasses

This example investigates the effect of enzymatic pre-treatment of sugar cane molasses on the ethanol yields.

The following enzyme blends were used:
Blend of Alpha-Amylase JA (26 FAU(F)/L); Glucoamylase TC (160 AGU/L and Protease ALC;
Blend of Alpha-amylase SC (18 KNU/L); Glucoamylase SF (112 AGU/L) and Protease ALC (0.048 AU/L fermentation medium)

Fresh sugar-cane molasses (°Bx about 80%) was pre-treated at 50° C. for 30 hours before fermentation using RED START″ yeast was carried out for 6 and 10 hours, respectively, at the same experimental conditions as indicated in Example 1.

### Results:

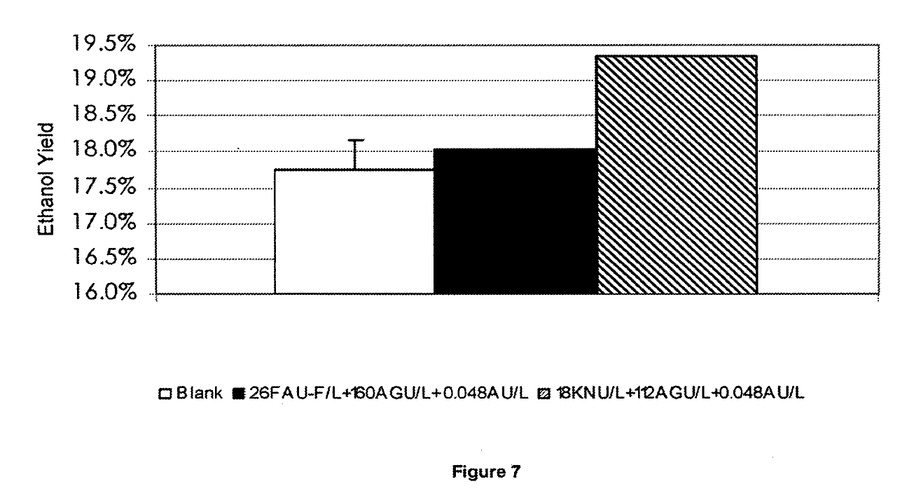

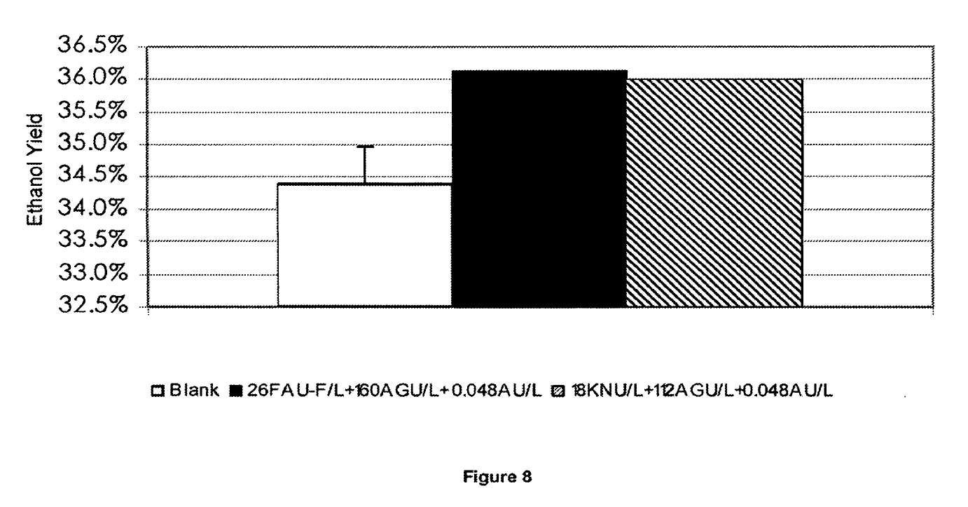

After 6 and 10 hours fermentation samples were taken for HPLC analyses. FIGS. 7 and 8, respectively, show the ethanol yields. A significant ethanol yield increase was found when pre-treating enzymatically at 50° C. for 30 hours before fermentation (confident level=95%).

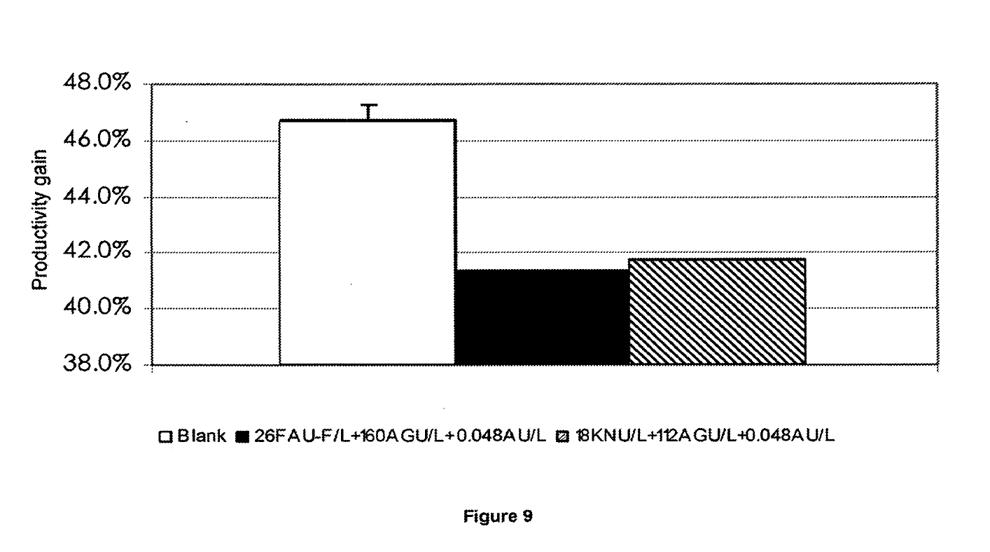

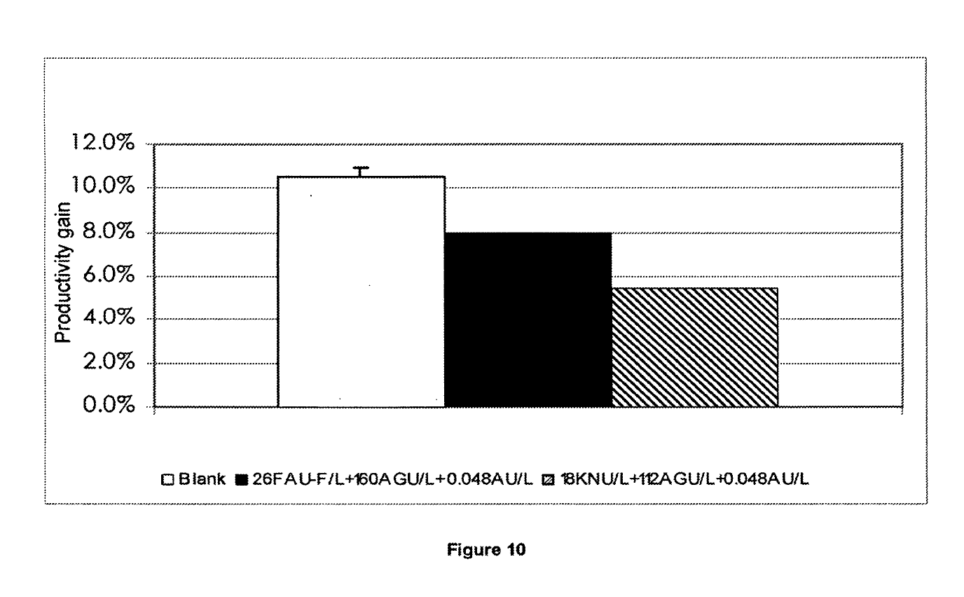

The productivity was estimated through the total reduction sugar (IRS) decay. TRS means the sum of dextrose and fructose obtained by HPLC analyses. FIGS. 9 and 10, respectively, show the TRS decay after 6 hours and 10 hours fermentation, respectively.

The productivity gain corresponds to the estimated gain of about 4% also found in Example 1.

In conclusion, enzymatic pre-treated of molasses at 50° C. for 30 hours leads to both yield increase and productivity improvements.

### Example 4

### Viscosity During Simultaneous Saccharification and Fermentation of Sugar Cane Molasses

This example investigates the viscosity of molasses during simultaneous saccharification and fermentation using below mentioned enzyme blends. The trails were carried out under the same conditions and using the same molasses as in Examples 1.

### Enzyme Blends:

Alpha-Amylase JA (26 FAU(F)/L)+Glucoamylase TC (160 AGU/L);

Alpha-amylase SC (9.6 KNU/L)+Glucoamylase SF (60 AGU/L)+Protease ALC (0.048 AU/L)

Alpha-amylase SC (19 KNU/L)+Glucoamylase SF (120 AGU/L)+Protease ALC (0.048 AU/L)

Alpha-amylase JA (13 FAU(F))+Glucoamylase TC (80 AGU/L)

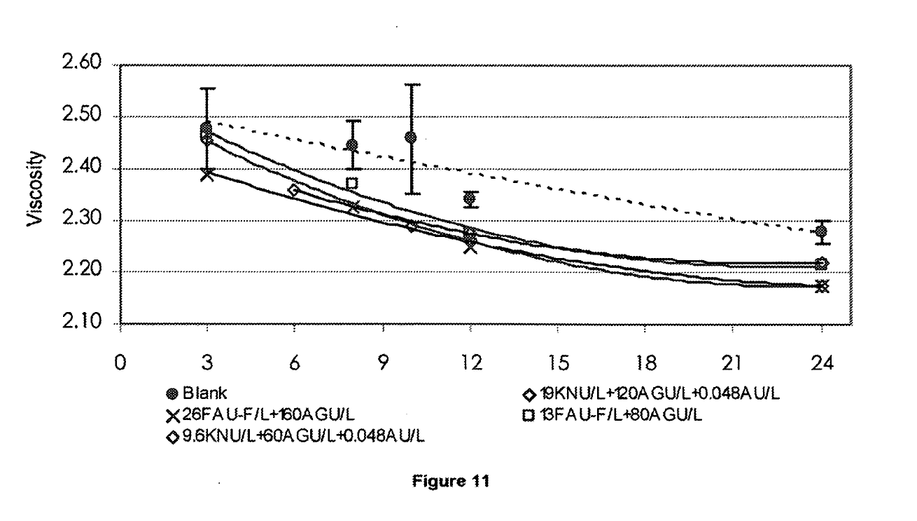

The viscosity was determined using a viscometer (ANTO PAAR, DMA 5000). The trail results are shown in FIG. 11.

### Example 5

### Simultaneous Saccharification and Fermentation of Sugar Cane Molasses in Industrial Scale Trial

This example investigates the effect of alpha-amylase and glucoamylase in large scale ethanol fermentation process using sugar-cane molasses as feedstock.

Fourteen test batches were carried out in industrial production scale in which a blend of enzymes was added. Twenty two blank batches at the same production scale were carried out. Test and blank batches were loaded with the same work volume (320 m³), as well as the same antibiotic and micronutrient dosage.

Yeast inoculum was obtained by recycling cell methodology in which whole fermenter broth passes through centrifuge separating liquid part—ethanol and water—solid part—yeast cell or yeast cream contenting at least 30% solids (corresponding to between 10⁷-10⁹ cells/mL fermentation medium) measured using a centrifuge (2500 rpm, room temperature for 10 minutes).

Yeast cream or inoculum is pre-treated with sulphuric acid concentrated up to 2.5-3.0pH and held under slightly agitation for 30 min. After that, the yeast cream is pumped into the fermenter. Inoculum volume is around 25% total fermenter work volume.

Fermentation broth or washed molasses is obtained through dilution of sugar-cane molasses storage to a Bx 75-80% in tap water to a Bx of 18-22% reaching 13-16% reducing sugar, that is continually pumped into fermenter according to a filling rate 40 m³/h, completing the operation in approx. 6 hours.

Tests batches received 9.6KNPU/L Alpha-amylase SC and 60AGU/LGlucoamylase SF, just before pumping the fermentation broth into the fermenter or just after having the inoculum in the fermenter. No enzymes were added into blank batches.

Fermentation temperature was 32±1.0° C. for all batches, including blanks and tests. No pH adjustment was done. However, samples of fermentation broth measured within 4.5-5.0pH.

Fermentation batches were finalized when the Bx measurement was stable between 6-8% and/or total reducing residual sugar was below 1%, typically within 8-10 h after starting the filling ramp.

The following analyses were carried out: pH (potentiometer), % Brix (refractometer), ethanol concentration (distillation and densitometry) and reducing sugar (Fehling titration). Fermentation yield was expressed through the conversion rate between ethanol formed during the fermentation (excluding ethanol carried by inoculum) by the total solids in the fermentation broth expressed through the Bx. Results are showed in the table 2 and 3.

Yield performance is summarized in the table 1:

TABLE 1
summary of performance of experiments

| Experiments | Mean | Variance |
|---|---|---|
| Blank (22 batches) | 38.38% | 0.0392% |
| Tests (14 batches) | 40.62% | 0.0524% |

T-test: two sample assuming unequal variances:
hypothesized mean difference for 95% probability

| | |
|---|---|
| P(T <= t) one tail | 0.002948 |

Conclusion: the means are 2.24% statistically different to 95% probability.

TABLE 2
Blank batches: raw data and yield calculation

| | | | | | | Beer (end of | | | |
|---|---|---|---|---|---|---|---|---|---|
| | Storage | | Fermentation | | Inoculum | fermentation) | | Ethanol | Yield |
| Blank | Molasses | | Broth | | Ethanol | | Ethanol | Real | oGL |
| Batches | Bx % | TRS % | Bx % | TRS % | oGL | TRRS % | oGL | oGL | real/Bx |
| BK_1 | 82.40 | 56.37 | 20.63 | 14.51 | 3.79 | 1.01 | 7.01 | 8.23 | 39.90% |
| BK_2 | 82.60 | 57.12 | 18.83 | 13.57 | 3.77 | 0.97 | 6.56 | 7.62 | 40.46% |
| BK_3 | 83.20 | 57.35 | 19.81 | 13.68 | 3.28 | 0.74 | 6.50 | 7.72 | 38.98% |
| BK_4 | 82.80 | 57.48 | 22.08 | 15.79 | 4.20 | 1.30 | 7.17 | 8.30 | 37.57% |
| BK_5 | 82.60 | 57.90 | 20.33 | 14.86 | 3.54 | 0.98 | 6.90 | 8.17 | 40.21% |
| BK_6 | 82.00 | 54.75 | 20.58 | 14.97 | 3.63 | 0.97 | 6.90 | 8.14 | 39.55% |
| BK_7 | 82.40 | 55.89 | 20.28 | 14.57 | 3.43 | 0.98 | 6.72 | 7.97 | 39.29% |
| BK_8 | 82.60 | 54.22 | 20.18 | 13.60 | 3.22 | 0.85 | 6.73 | 8.06 | 39.95% |
| BK_9 | 83.20 | 55.06 | 21.94 | 14.92 | 3.60 | 0.90 | 7.32 | 8.73 | 39.80% |
| BK_10 | 82.60 | 54.66 | 21.97 | 14.64 | 4.21 | 1.00 | 7.18 | 8.31 | 37.81% |
| BK_11 | 82.60 | 55.76 | 20.92 | 14.82 | 3.39 | 1.14 | 6.64 | 7.87 | 37.63% |
| BK_12 | 81.00 | 55.12 | 20.96 | 14.54 | 3.64 | 0.97 | 7.01 | 8.29 | 39.54% |
| BK_13 | 80.00 | 54.47 | 20.97 | 14.78 | 3.53 | 0.99 | 6.94 | 8.23 | 39.26% |
| BK_14 | 79.60 | 53.44 | 13.86 | 9.47 | 1.71 | 0.55 | 4.09 | 4.99 | 36.02% |
| BK_15 | 79.20 | 55.64 | 15.75 | 11.91 | 1.88 | 0.54 | 4.58 | 5.60 | 35.58% |
| BK_16 | 79.40 | 54.37 | 17.55 | 14.61 | 3.54 | 4.11 | 5.17 | 5.79 | 32.98% |
| BK_17 | 79.00 | 54.52 | 16.52 | 10.75 | 2.77 | 0.71 | 4.98 | 5.82 | 35.22% |
| BK_18 | 79.60 | 54.58 | 19.30 | 15.03 | 2.50 | 0.74 | 5.81 | 7.07 | 36.61% |
| BK_19 | 79.60 | 54.27 | 19.80 | 15.90 | 2.98 | 0.89 | 6.53 | 7.88 | 39.78% |
| BK_20 | 78.40 | 54.27 | 19.78 | 14.81 | 3.43 | 1.05 | 6.67 | 7.90 | 39.93% |
| BK_21 | 78.60 | 53.60 | 18.73 | 14.00 | 3.14 | 0.93 | 6.12 | 7.25 | 38.71% |
| BK_22 | 79.40 | 55.73 | 19.90 | 14.11 | 2.82 | 0.63 | 6.49 | 7.88 | 39.61% |

Bx % or Bx - total solid dissolved in solution (fermentation broth or fermentation beer)
TRS %—total reducing sugar
Ethanol oGL - concentration of ethanol (% v/v)
TRRS %—total reducing residual sugar
Ethanol Real oGL - concentration ethanol effectively formed during the fermentation or excluding ethanol carried by inoculum
Yield % - oGLreal/brix - percentation of conversion between ethanol formed during the fermentation by the total solid expressed by Bx %

TABLE 3
Test batches: raw data and yield calculation

| | | | | | | Beer (end of | | | |
|---|---|---|---|---|---|---|---|---|---|
| | Storage | | Fermentation | | Inoculum | fermentation) | | Ethanol | Yield |
| Tests | Molasses | | Broth | | Ethanol | | Ethanol | Real | oGL |
| Batches | Bx % | TRS % | Bx % | TRS % | oGL | TRRS % | oGL | oGL | real/Bx |
| Te_1 | 79.40 | 54.37 | 17.55 | 15.98 | 4.18 | 4.22 | 5.88 | 6.52 | 37.18% |
| Te_2 | 79.40 | 54.37 | 17.55 | 15.75 | 3.28 | 0.97 | 5.66 | 6.56 | 37.39% |
| Te_3 | 79.20 | 52.33 | 16.40 | 11.36 | 2.34 | 0.69 | 5.30 | 6.42 | 39.16% |
| Te_4 | 79.20 | 52.33 | 16.40 | 11.38 | 1.90 | 0.63 | 5.20 | 6.45 | 39.34% |
| Te_5 | 79.20 | 54.49 | 19.40 | 15.02 | 1.98 | 0.80 | 6.12 | 7.69 | 39.64% |
| Te_6 | 79.20 | 54.49 | 19.30 | 15.20 | 2.10 | 0.75 | 6.16 | 7.70 | 39.90% |
| Te_7 | 79.20 | 55.00 | 19.70 | 15.40 | 2.80 | 1.74 | 6.36 | 7.71 | 39.14% |
| Te_8 | 79.20 | 55.00 | 20.00 | 15.54 | 2.92 | 1.02 | 6.89 | 8.40 | 41.98% |
| Te_9 | 78.40 | 54.27 | 20.30 | 15.26 | 3.70 | 0.78 | 7.26 | 8.61 | 42.42% |
| Te_10 | 78.40 | 54.27 | 19.80 | 14.75 | 3.62 | 0.79 | 7.26 | 8.64 | 43.64% |
| Te_11 | 78.60 | 53.60 | 18.10 | 13.55 | 3.22 | 0.63 | 6.04 | 7.11 | 39.28% |
| Te_12 | 78.60 | 53.60 | 18.30 | 14.00 | 3.02 | 0.58 | 6.42 | 7.71 | 42.13% |
| Te_13 | 79.40 | 55.73 | 19.60 | 15.22 | 2.90 | 0.58 | 7.10 | 8.69 | 44.35% |
| Te_14 | 79.40 | 55.73 | 19.90 | 15.30 | 2.76 | 0.60 | 6.98 | 8.58 | 43.12% |

Bx % or Bx - total solid dissolved in solution (fermentation broth or fermentation beer)
TRS %—total reducing sugar
Ethanol oGL -3 concentration of ethanol (% v/v)
TRRS %—total reducing residual sugar
Ethanol Real oGL - concentration ethanol effectively formed during the fermentation or excluding ethanol carried by inoculum
Yield % - oGLreal/brix - percentation of conversion between ethanol formed during the fermentation by the total solid expressed by Bx %

1. A process for producing ethanol from molasses using a fermenting organism, wherein molasses is
i) treated with a combination of alpha-amylase and glucoamylase, and
ii) fermented using one or more fermenting organisms at a cell count in the range from 10⁷-10¹⁰ cells/mL fermentation medium.

2. The process of claim 1, wherein the cell count is in the range 10⁸-10¹⁰ cells/mL fermentation medium.

3. The process of claim 1, wherein enzyme treatment in step i) and fermentation in step ii) are carried out sequentially or simultaneously.

4. The process of claim 1, wherein a step i) is carried out as a pre-treatment step at conditions suitable for the enzymes.

5. The process of claim 1, wherein step i) is carried out at a temperature in the range from 20-70° C.

6. The process of claim 1, wherein the pH during treatment in step i) is in the range from 4-6.

7. The process of claim 1, wherein step i) is carried out by subjecting molasses to enzyme treatment for 1-10 days.

8. The process of claim 1, wherein fermentation in step ii) or simultaneous steps i) and ii) are carried out for between 1 and 96 hours.

9. The process of claim 1, wherein enzyme treatment in step i) and fermentation in step ii) are carried simultaneously.

10. The process of claim 9, wherein the temperature during simultaneous step i) and step ii) is optimal to the fermenting organism.

11. The process of claim 10, wherein the temperature is in the range from 25-60° C.

12. The process of claim 11, wherein simultaneous step i) and step ii) is carried out at a temperature between 25 and 40° C. when the fermenting organism is yeast.

13. (canceled)

14. The process of claim 1, wherein the alpha-amylase is an acid fungal alpha-amylase derived from a strain of Aspergillus, Rhizomucor or Meripilus.

15. The process of claim 1, wherein the glucoamylase is selected from the group consisting of glucoamylases derived from the genera Aspergillus, Athelia, Talaromyces, Rhizopus, Humicola, and Trametes.

16. The process of claim 1, further wherein the fermenting organism is yeast and is subjected to one or more proteases during fermentation in step ii) or simultaneous enzyme treatment and fermentation.

17. The process of claim 16, wherein the protease is of fungal or bacterial origin.

18. The process of claim 17, wherein the fungal protease is derived from a strain of the genus Aspergillus, or a strain of Rhizomucor.

19. The process of claim 17, wherein the protease is derived from a strain of Bacillus.

20. The process of claim 1, wherein the molasses is sugar cane molasses.

21. The process of claim 1, wherein the fermenting organism is yeast.
